(19) United States
(12) Patent Application Publication
(10) Pub. No.: US 2005/0211192 A1
(43) Pub. Date: Sep. 29, 2005

(54) Animal cover having a temperature altering device

(76) Inventor: Ali Nilforushan, Rancho Santa Fe, CA (US)

Correspondence Address: CATALYST LAW GROUP, APC, 9710 SCRANTON ROAD, SUITE S-170, SAN DIEGO, CA, 92121, US

(21) Appl. No.: 10/807,695

(22) Filed: Mar. 24, 2004

(51) Int. Cl. A61K 31/495

(52) U.S. Cl. 119/850

(57) ABSTRACT

A device useful for delivering a temperature altering regimen to a specific and defined location on an animal's body, and thus altering the temperature of an animal's body, or defined area thereof. It is desirable to alter the temperature of an animal's body for a variety of reasons, including: maintaining a desirable basal body temperature in an extreme climate; maintaining basal body temperature to treat an illness affecting said temperature; providing a temperature greater or lesser than basal temperature to muscle groups and/or joints to help with swelling and aching following a strenuous workout; providing a temperature greater or lesser than basal temperature to joints to assist with pain associated with swelling due to arthritis; and to lower the temperature of a body following a strenuous workout.

### FIELD OF THE INVENTION

This invention relates generally to the field of animal care, and particularly to delivering a temperature altering regimen to a specific and defined location on an animal's body.

### BACKGROUND

Covers, blankets, clothing and other articles adapted for wearing by an animal have long been used. Common examples include, clothing for dogs, fly protectors (used to prevent or lessen damage to cattle and other farm animals caused by ectoparasites, such as flies and mosquitoes) and horse blankets. Conventional horse blankets are provided to protect a horse from mud, dirt, and moisture, and to provide a degree of thermal insulation. These conventional horse blankets typically conform to the shape of a horse's upper body to provide adequate protection from the elements.

The horse blanket of the prior art provides a general protection from the elements; however, the prior art is deficient in providing targeted, extreme temperature to an area of the horses body. Similarly, other animal covers and clothing merely provide a general barrier between the animal and the elements. Furthermore, the animal covers of the prior art are not useful for delivering an extreme temperature regimen to the animal's body, or to defined area thereof. Thus, there is a need in the art for an animal cover that delivers targeted and extreme temperature to an area of an animal's body, or to the entire body.

### SUMMARY OF THE INVENTION

One embodiment of the present invention relates to a device useful for delivering a temperature altering regimen to a specific and defined location on an animal's body. The invention is useful for altering the temperature of an animal's body, or specific area thereof. It is desirable to alter the temperature of an animal's body for a variety of reasons, including: maintaining a desirable basal body temperature in an extreme climate; maintaining basal body temperature to treat an illness affecting said temperature; providing a temperature greater or lesser than basal temperature to muscle groups and/orjoints to help with swelling and aching following a strenuous workout; providing a temperature greater or lesser than basal temperature to joints to assist with pain associated with swelling due to arthritis; and to lower the temperature of a body following a strenuous workout. Thus, the current invention is useful in a variety of fields, including farming, veterinary, competition animals and home use.

In one particular aspect of the present invention, is a horse blanket used to deliver a temperature altering regimen to a competition horse. The invention comprises strategically located cavities further comprising the temperature altering device. The horse blanket, therefore, places the temperature altering device in a specific and defined location on the horse's body wherein a temperature altering regimen is delivered.

In one embodiment of the method, the invention is brought to a desired temperature and is utilized to deliver a temperature altering regimen to the body of an animal.

### BRIEF DESCRIPTION OF THE DRAWINGS

FIG. 1A shows the horse blanket embodiment of the current invention; wherein the horse blanket is laid open.

FIGS. 1B-D illustrates alternative configurations for placing the cavity on a horse blanket. The cavity further comprises a temperature altering device, thus their placement will define the region of the blanket that will deliver temperature altering regimen.

FIG. 2A is a cross sectional view of a cavity embedded into a horse blanket.

FIG. 2B is a view of one embodiment for placement of an adjustable cavity in a horse blanket, specifically in this illustration on the flap of the horse blanket body.

FIG. 2C is a view of one alternative embodiment for placement of an adjustable cavity in a horse blanket, specifically in this illustration on the flap of the horse blanket body.

FIG. 3 is a cross sectional view of a cavity having a sealable pocket mouth for removably placing a temperature altering device.

### DETAILED DESCRIPTION OF THE INVENTION

Abbreviations and Terms

The current invention is a cover used to alter the temperature of a user. Preferably, the user referred to herein is an animal, more preferably the user is a competition animal and most preferably, the animal is a competition horse. For the remainder of this description of the current invention, a horse blanket will be described; however, those of ordinary skill in the art will readily adapt the current invention to a variety of covers for a variety of animals. These uses of the current invention are well with in the scope of the current invention.

It is desirable to alter that temperature of an animal for a variety of reasons. Veterinarians typically use ice baths to lower the temperature of an animal with a fever. Similarly, farmers and other animal owners, particularly those in more extreme climates, desire to alter the temperature of their animals. Animal blankets are frequently used on animals in extremely cold climates to keep these animals warm. Competition animals exert maximum effort during a performance or practice, and as a result, will often develop sore muscles, inflamed joints, increased body temperature and a variety of other effects. Such competitions animals include, but are not limited to greyhound race dogs, thorough bread race horses, sport horses (e.g., jumping, driving, dressage, vaulting and endurance) and agility dogs.

The current invention is directed towards a new device and method for delivering a temperature altering regimen to an animal. In the preferred embodiment, the animal is a competitions horse. The device in this embodiment is a cover, similar to a horse blanket that covers the body of the horse and delivers a temperature altering regimen directly to a specific location on the horse's body. The device of the current invention is adjustable, allowing the temperature altering regions to precisely fit a variety of differently shaped and sized horses on a variety of body areas. The device and method is further described below with reference to the attached drawings.

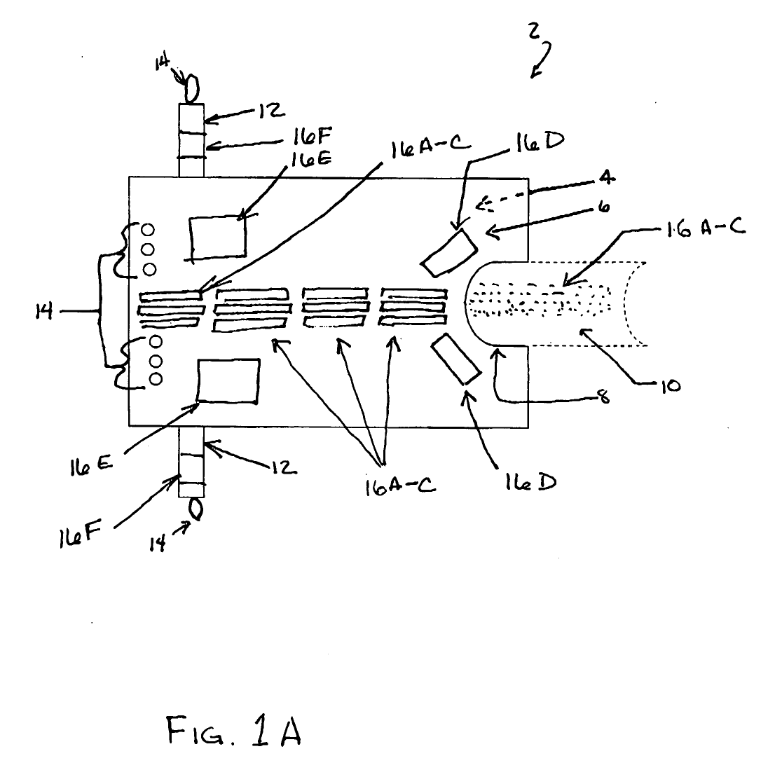

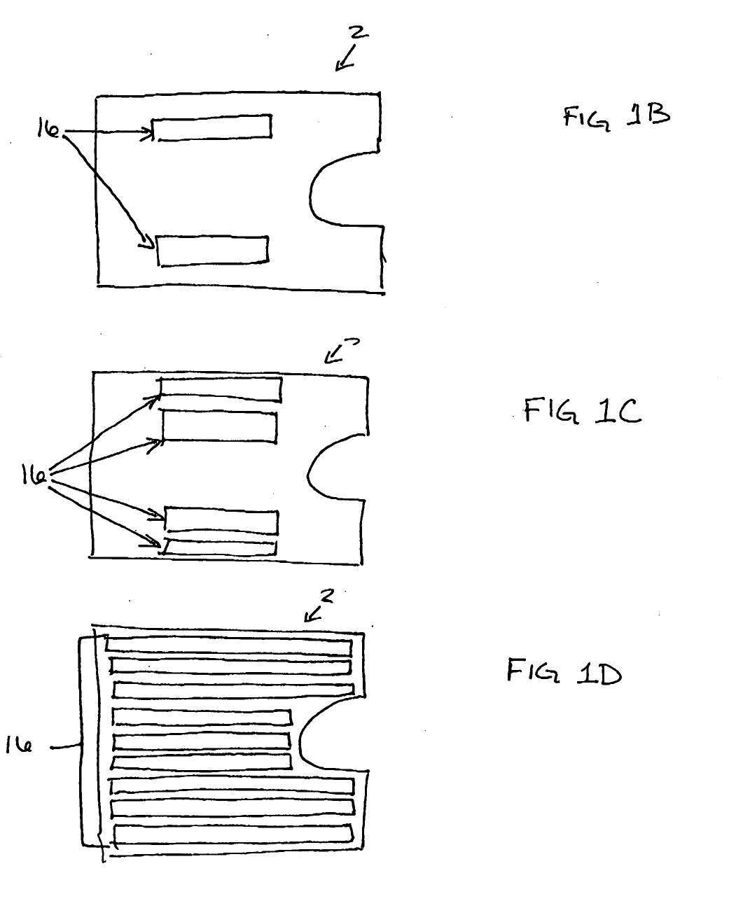

FIG. 1A is a horse blanket 2, having an interior side 4 and an exterior side 6. The interior side 4 refers to the side of the blanket that is closest to the horse's body. In the preferred embodiment, the material of horse blanket 2 is constructed from a material that will wick moisture away from the body of the horse, thereby preventing rash and other associated problems. Other materials can be used within the spirit of the current invention.

The front end of the horse blanket comprises a cut away portion 8 designed to fit around the horse's neck, resting just above the withers. Alternatively seen as a broken line, the horse blanket comprises a protrusion 10 that covers a majority of the horse's neck, stopping just short of the horse's head. The rear sides of the horse blanket comprises flaps 12, which are fitted around the hp of the horse and a releasably attached to connectors 14 at the rear of the horse blanket. The horse blanket is attached to the horse's body using fastening systems well known in the art.

It is to be understood, that the preferred embodiment wherein a horse blanket comprises the invention is by way of example only. Furthermore, horse blankets are well known in the art, and the description of a horse having a cut away 8 exposing the horse's neck, or a protrusion 10 covering the horse's neck are mere descriptions of a horse blanket. Those of ordinary skill in the art will readily adapt the current invention to fit a variety of covers beyond blankets, and the covers comprising the current invention are useful on more animals that just horses. These variations are within the scope of the current invention.

Also shown in FIG. 1A is a plurality of cavities 16. The cavities 16, hold the temperature altering device (not shown) at a determined position within the horse blanket 2. Cavities 16a-c are strategically located within blanket 2 to contact the horse's spine 16a and spinal muscles 16b-c. Cavities 16d are strategically located within blanket 2 to contact the horse's shoulder muscles. Cavities 16e are strategically located within blanket 2 to contact the horse's hip muscles. Cavities 16f are strategically located within blanket 2 to contact the horse's stifle joint. In an alternative embodiment wherein the horse blanket further comprises a protrusion 10 covering the horse's neck, cavities 16a-c may also extend through the protrusion 10 making contact with the horse's c-spine and associated muscles.

Cavities 16 can be located in any portions of horse blanket 2 that is desired. For example, in FIG. 1B, the cavities are shown strategically located within the horse blanket 2 to contact the horse's thoracic cavity. If the horse blanket 2 is designed such that it is capable of attaching around the underside of a horse, then the cavities 16, can be strategically located to contact the horse's girth and abdomen, as is shown in FIG. 1C. In a further embodiment of the current invention, the cavities 16, are located throughout the entire horse blanket (FIG. 1D). These and other embodiments for locating these cavities in an animal cover are well within the scope of this current invention.

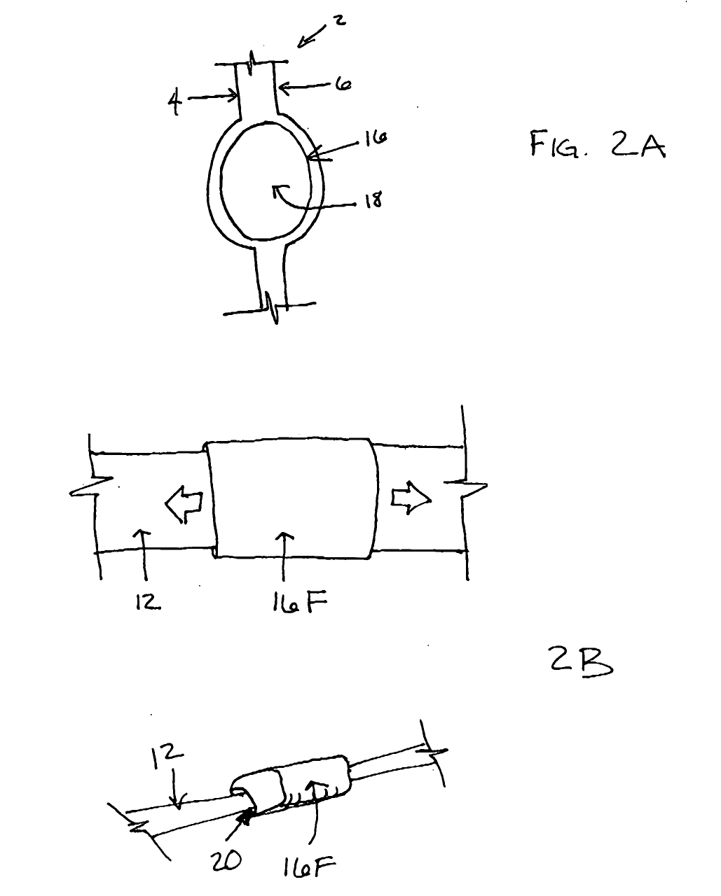

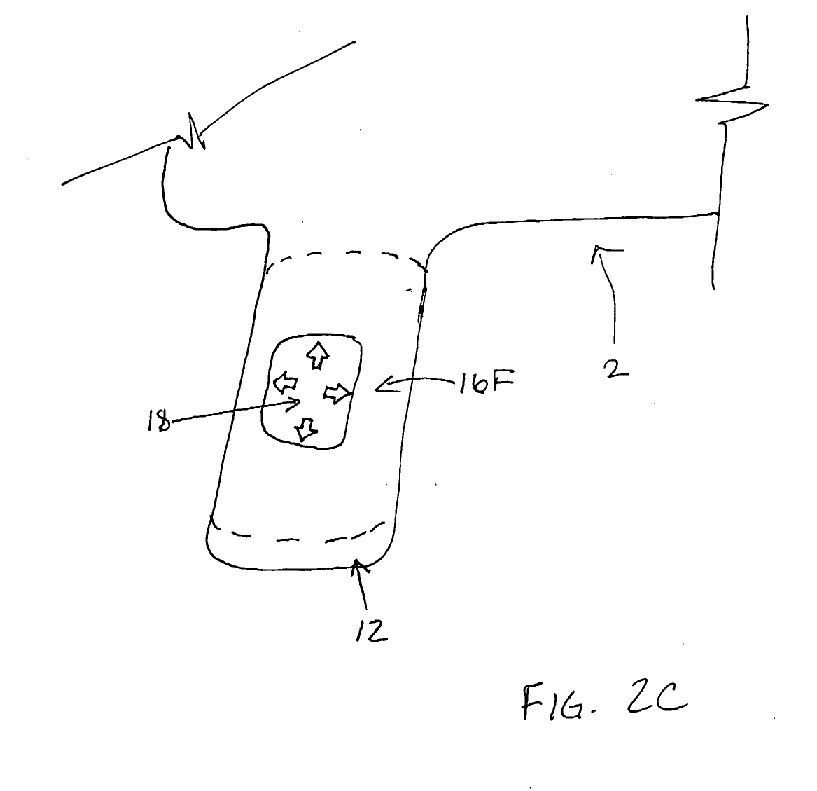

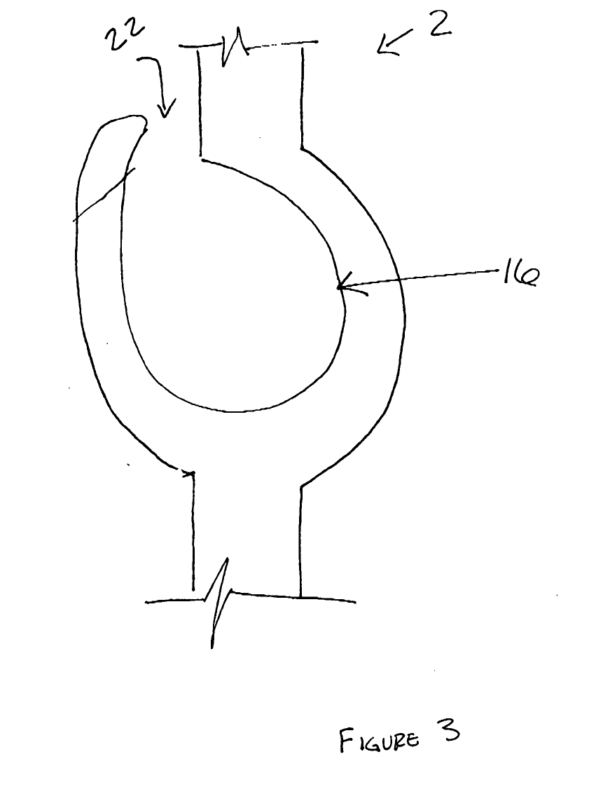

Cavities 16 are designed to hold the temperature altering device of the current invention. In FIG. 2A, a cavity of the current invention is illustrated in cross sectional view. Horse blanket 2, comprises interior side 4; exterior side 6 and cavity 16. Preferably, horse blanket 2, is made of a material that wicks moisture away from the horse's body. The material of the horse blanket 2 forming cavity 16; however, preferably reflects the temperature from the temperature altering device 18 towards the body of the horse, thereby delivering an efficient temperature altering regimen to that location of the body. In this aspect, cavity 16 is surrounded by a temperature reflective material on the exterior 6 side of the horse blanket 2, and a temperature permeable material on the interior 4 side of horse blanket 2.

Alternatively, cavities 16 are designed such that they sit on horse blanket 2. This alternative embodiment is useful for delivering cavities 16 that are easily positioned and re-positioned in a variety of locations. Cavity 16f, located on the flap 12 of horse blanket 2 is an example of such a cavity 16. Flap 12 is designed to form part of horse blanket 2 that wraps around the thigh of a horse. This optional flap 12 is useful for securing the horse blanket on a horse. A temperature altering device can be placed in or on this flap 12, and deliver a temperature altering regimen to the stifle joint, inner thigh, groin and/or other horse anatomy positioned in that region.

Flap 12 will generally extend from horse blanket 2 near the front of the horse's thigh/point of hip region. Flap 12 can be placed behind the horse's thigh and releasably connect to the horse blanket near the rear of thigh/buttock region of the horse. Preferably, the releasable connection 14 is a spring clamp and loop assembly; however, a variety of other connector mechanisms is well known in the art and can be used in this embodiment. The connector 14 is also adjustable, allowing for a conforming proper fit for flap 12 with a variety of sized and shaped horses. As used with the current invention, flap 12, comprises a cavity 16f for delivering a temperature altering regimen to a horse.

In the preferred embodiment, cavity 16f is adjustable along flap 12 for proper positioning of the temperature altering device 18. In this embodiment, shown in FIG. 2B, the cavity 16f is formed of material separate from the interior side 4 and exterior side 4 of the horse blanket 2. Cavity 16f is slideably connected to flap 12, thereby allowing for adjustable positioning of the temperature altering device 18 on the horse. Cavity 16f can be crafted in a variety of ways, including, but not limited to having an added layer of material forming a shaft to allow for sliding along flap 12. In an alternative embodiment, the cavity 16f is formed within flap 12 as described above for cavities 16a-e; and cavity 16f is much larger that temperature altering device 18 (FIG. 2C). In this alternative embodiment, the temperature altering device is moveable along flap 12, and is secured at a desired position using a clamping mechanism. These and other embodiments will be apparent to those of ordinary skill in the art and do not exceed the current invention. In addition, the positional cavity and/or temperature altering device is not limited to that on flap 12, as is apparent to those of ordinary skill in the art.

Temperature altering device 18 delivers a desired temperature to a specific area of a horse's body. The delivered temperature can be either greater or lesser than horse's current core temperature. Following a strenuous work out the horse's body will increase its temperature relative to basal temperature. In this instance, the current invention comprises a cooling temperature altering device 18 can be applied to the horse's body to help the horse cool down. Horse's muscles and joints will often require heat or cold, and this is particularly true following a strenuous work out or an injury. In theses instances, the current invention comprises either a heating or a cooling temperature altering device 18.

The temperature altering device 18 is located within cavity 16. The temperature altering device 18 can either be permanently located within cavity 16 or removably located within cavity 16. If the temperature altering device 18 is permanently located within cavity 16, then it can be sown within the fabric, or otherwise included within. If; however, the temperature altering device 18 is removably located within cavity 16, then cavity 16 can be in the form of a pocket, having a securable pocket mouth 22 (FIG. 3). Pocket mouth 22 can either be on the interior side 4 of the exterior side 6 of horse blanket 2. In the preferred embodiment, pocket mouth 22 is located on exterior side 6 of horse blanket 2, thereby allowing for easy placement and removable of temperature altering device 18 while horse blanket 2 is on the horse's body.

Securable pocket mouth 22, can be closed using a variety of means well known in the art. It is preferable, that the securing means for securable pocket mouth 22 is such that the entire length of securable pocket mouth 22 is sealed, thereby preventing a loss of temperature from temperature altering device 18. Securable pocket mouth 22 can be; hook and loop, snap, zipper, button, tuck, or a variety of other means well know in the art.

Temperature altering device 18 is brought to a desired temperature; is strategically placed on the horse's body; and will deliver a temperature altering regimen to that specific area of the horse's body. When temperature altering device 18 is permanently affixed within the horse blanket 2, then the entire horse blanket 2 must be subjected to a means for bringing temperature altering device 18 to a desired temperature. In one example for accomplishing this goal, the temperature altering device is a series of electrically connected heating elements having a plug mechanism on one end. The plug is electrically connected to a source and will cause the temperature of the heating elements to rise. The heating elements can be brought to a desired temperature before the horse blanket 2 is placed on the horse, or the horse blanket 2 can remain plugged in while on the horse. A cooling horse blanket 2 can similarly have cooling elements electrically connected and having a plug member at one end.

Alternatively, with a horse blanket 2 comprises a permanent temperature altering device 18, the temperature altering device 18 comprises a material that is capable of being adjusted to a desired temperature, and which will hold said temperature. Such materials are well know in the art and are generally referred to as heat packs or cool packs. Some examples of such heat and cooling pack include, without limitation: the Hot & Cold Flexible Gel Pack by 3M, Minneapolis, Minn.; and the Hot/Cold Reusable Gel Pack by Accu-Therm, Taipei, Taiwan. It is well known in the art, that these reusable gel materials are capable of obtaining a desired temperature, and then can be used to deliver that temperature to a part of the body. The temperature of these materials is generally altered using refrigeration, ovens, submersion in hot or cold water; microwaves and like methods.

With the current invention wherein said horse blanket 2 comprises a permanent temperature altering device 18, and the temperature altering device 18 further comprises a gel similar to that used in a hot pack or cool pack, the entire horse blanket 2 must be exposed to the external temperature. In this embodiment, the refrigeration, oven or microwave means are preferable. Submersion in hot or cold water is not desires, though it is an option, because the entire horse blanket 2 will become wet. The horse blanket 2 is placed in a heating or cooling environment; the heating or cooling environment will activate the temperature altering device 18 to reach a desired temperature; the horse blanket 2 with activated temperature altering device is placed on the horse strategically locating the temperature altering device 18 near a specific area of the horse's body, and a temperature altering regimen will take place. Should temperature altering device return to a temperature that is no longer useful for the temperature altering regimen, then the necessary steps should be repeated to bring temperature altering device 18 to a desired temperature and, thus, continue delivery of a temperature altering regimen. Suitable materials and devices useful for delivering a temperature altering regimen will be obvious to use in conjunction with the current invention, and one of ordinary skill in the art will readily do so without exceeding the scope of the current invention.

Horse blanket 2 can comprise temperature altering device 18 removably located in cavity 16. In this embodiment, the temperature altering device 18 can be a heating or cooling element, a gel pack similar to that used in heat and cool packs, or other similar removable device for altering temperature. Similar to the above description, these temperature altering devices can be electrically connected to a power source (for the heating or cooling element) or can be placed in a temperature altering environment, brought to a desired temperature and then returned to horse blanket 2 to deliver a temperature altering regimen. If a heat pack or cool pack is used, then submersion in hot or cold water is a means for adjusting the temperature of said temperature altering device 18, because only the packs and not the whole blanket will be submerged in water. Also, in this embodiment, temperature altering device 18 can be a single use device or material. For example, cavity 16 can be filled with ice, or with an activated disposable heat pack (e.g., those containing sodium chloride and water for heat, or those containing ammonium nitrate and water for cold). Suitable materials and devices useful for delivering a temperature altering regimen will be obvious to use in conjunction with the current invention, and one of ordinary skill in the art will readily do so without exceeding the scope of the current invention.

If the temperature altering device 18 is removably located in cavity 16, then said temperature altering device 18 is removed from the cavity through sealable pocket mouth 22; is brought to a desired temperature; is returned to cavity 16; sealable pocket mouth 22 is sealed; and a temperature altering regimen is delivered to strategic locations of the horse's body. Depending on whether sealable pocket mouth 22 is located on the interior side 4 or exterior side 6 of horse blanket 2, will determine ease of removing and replacing temperature altering device 18 from cavity 16 when the horse blanket 2 is on the horse. When sealable pocket mouth 22 is on the exterior side 6 of horse blanket 2, then the temperature altering device can easily be removed and replaced from cavity 16 when horse blanket 2 is on the horse. More difficult, is removing and replacing temperature altering device when sealable pocket mouth 22 is located on the interior side 4 of horse blanket 2 when the horse blanket 2 is on the horse. Cavities 16a-c located at or near the spine and spinal muscles of the horse may be impossible to access when the horse blanket 2 is on the horse. Thus, it is preferable that sealable pocket mouth 22 is located on the exterior side 6 of horse blanket 2. Alternatively, if sealable pocket mouth 22 is on the interior side 4 of horse blanket 2, it is preferable that the activated temperature altering device is placed within cavity 16 and then horse blanket 2 is placed on the horse's body.

Various modifications and alterations of the invention will become apparent to those skilled in the art without departing from the spirit and scope of the invention, which is defined by the accompanying claims. For example, it should be noted that steps recited in any method claims below do not necessarily need to be performed in the order that they are recited. Those of ordinary skill in the art will recognize variations in performing the steps from the order in which they are recited. For example, in certain embodiments, steps may be performed simultaneously. The accompanying claims should be constructed with these principles in mind.

1. An animal cover adapted to conform to the shape of an animal's body to deliver a temperature to a specific targeted area of the body, the animal cover comprising:
a. a body of the animal cover having an interior and exterior side;
b. a plurality of cavities strategically located within the body of the animal cover to deliver a temperature altering regimen to a targeted area of the animal's body; and
c. a temperature altering device.

2. The animal cover of claim 1, wherein the plurality of cavities are strategically located within the body of said animal cover to deliver a temperature altering regimen to the animal's muscle groups selected from the group consisting of: spine, shoulder, neck, thigh and hip.

3. The animal cover of claim 1, wherein the plurality of cavities are strategically located within the body of said animal cover to deliver a temperature altering regimen to the animal's joints selected from the group consisting of: stifle, hip, shoulder and elbow.

4. The animal cover of claim 1, wherein the plurality of cavities are strategically located within the body of said animal cover to deliver a temperature altering regimen to an area of the animal's anatomy selected from the group consisting of: neck, withers, spine, scapula, back, loin, croup, thigh, buttock, stifle, abdomen, girth, elbow, forearm, arm, chest, shoulder and neck.

5. The animal cover of claim 1, wherein the temperature altering device is located within the plurality of cavities.

6. The animal cover of claim 5, wherein the temperature altering device is permanently located in the plurality of cavities.

7. The animal cover of claim 5, wherein the temperature altering device is removably located in the plurality of cavities.

8. The animal cover of claim 7, wherein the plurality of cavities form a sealable pocket for holding the temperature altering device, said sealable pocket further comprising a seal selected from the group consisting of: hook and loop, zipper, buttons and snaps.

9. The animal cover of claim 1, wherein the temperature altering device is brought to a desired temperature by placing the entire animal cover in a refrigerated or heated environment until said temperature altering device reaches a desired temperature and can be used to deliver a temperature altering regimen to an animal.

10. The animal cover of claim 1, wherein the temperature altering device is removed from said animal cover and is brought to a desired temperature by placing said temperature altering devices in a refrigerated or heated environment until said temperature altering device reaches a desired temperature and can be returned to said animal cover and used to deliver a temperature altering regimen to an animal.

11. The animal cover of claim 1, wherein the body of the animal cover is made of a material that will wick moisture away from the body of the animal.

12. The animal cover of claim 1, wherein the plurality of cavities further comprise a material on the exterior side of the body of said animal cover that will reflect the temperature emitted from the temperature altering device towards the body of the animal for maximum efficiency of temperature transfer.

13. The animal cover of claim 1, wherein the plurality of cavities are adjustable about the body of the animal cover for optimal placement on a variety of differently sized animals.

14. The animal cover of claim 13, wherein the cavity for delivering a temperature altering regimen to a stifle is located on a flap of the body of said animal cover, said flap wraps from the outer thigh around to the inner thigh and is adjustably attached to the buttock/croup area of the animal cover.

15. The animal cover of claim 14, wherein the cavity for delivering a temperature altering regimen to the stifle is adjustable about the flap of the body of the animal cover, allowing for optimal positions of the temperature altering device between the stifle joint and the abdomen/groin area of the animal.

16. The animal cover of claim 1, wherein the animal is selected from the group consisting of horse, dog; llama, sheep and cat.

17. The animal cover of claim 1, wherein said animal cover is a horse blanket and the animal is a horse.

18. A method for delivering a temperature altering regimen comprising the steps of:
a. altering the temperature of a temperature altering device located within the body of an animal cover having a temperature altering device and being designed and fitted to deliver a temperature altering regimen to specific areas of an animal's body;
b. placing the animal cover on the body of an animal; and
c. allowing a temperature altering regimen to run its course.

19. The method of claim 18, wherein the step of altering the temperature of an animal cover comprises placing the entire cover into a refrigeration or heating environment and allowing the temperature altering device to reach a desired temperature.

20. The method of claim 18, wherein the step of altering the temperature of an animal cover comprises removing the temperature altering device from said animal cover, placing said temperature altering device in to a refrigeration or heating environment, allowing said temperature altering device to reach a desired temperature, and replacing said temperature altering device into said animal cover.

21. The method of claim 18, wherein the step of placing the animal cover on the body of an animal comprises adjusting and properly aligning said animal cover to allow for the optimally strategic alignment of the temperature altering device with an area of the animal's body to receive a temperature altering regimen.

22. The method of claim 21, wherein the step of placing the animal cover on the animal further comprises wrapping the animal cover around the outer thigh and the inner thigh of the animal to optimally and adjustably align a temperature altering device between the stifle and the groin of the animal's body.

23. The method of claim 18, wherein the step of allowing the temperature altering regimen to run its course comprises letting the temperature altering device deliver temperature to the animal's body for an optimally defined period of time.

24. The method of claim 23, wherein the step of allowing the temperature altering regimen to run its course further comprises repeating the steps of altering the temperature of the animal cover and placing the animal cover on the body of an animal.

25. The method of claim 18, wherein the animal is selected from the group consisting of horse, dog; llama, sheep and cat.

26. The animal cover of claim 18, wherein said animal cover is a horse blanket and the animal is a horse.
